SEQUENCE LISTING (13 sequences)

SEQ ID NO: 1 - DNA, 1152 bp, Bacillus subtilis strain ARRMK-33
atgaatcattcaaaaacacttttgttaaccgcggcagccggattgatgctcacatgcggtgcggtttcttcccaggccaagcataagctgtctgatccttatcattttaccgtaaatgcggcggcggaaacggaaccggttgatacagcaggtgatgcagctgatgatcctgcgatttggctggatcccaagaatcctcagaacagcaaattgatcacaactaataaaaaatcaggcttagtcgtgtacagcctagagggaaagatgcttcattcctatcctaccgggaagctgaacaatgttgatatccgctatgattttccgttgaacggaaaaaaagtcgatattgcggcggcatccaatcggtctgaaggaaagaataccattgagatttacgccatcgacggaaaaaacggcacattacaaagcattacggatccagaccgcccgattgcgtcagcaatagatgaagtatacggtttcagcttgtaccacagtcaaaaaacaggaaaatattacgcgatggtgacagggaaagaaggcgaatttgaacaatacgaattaaatgctgataaaaatggatacatatccggcaaaaaggtaagggcgtttaaaatgaattctcagacagaagggatggcagcagacgatgaatacggcagtctttatatcgcagaagaagatgaggccatctggaagttcagcgctgagccggacggcggcagtaacggaacggttatcgatcgtgccgacggcaggcatttaacccctgatattgaaggactgacgatttactacgctgctgacgggaaaggttatttgcttgcatcaagccagggtaacagcagctacgcgatttatgaaagacagggacagaacaaatatgttgcggactttcagataacagacgggcctgagacagacggcacaagcgatacagacggaattgacgttctgggtttcgggctgggacctgaatatccgttcggcttttttgtcgcacaggacggagaaaatatagatcacggccaaaaggccaatcaaaattttaaaatggtgccttgggaaagaatcgctgataaaatcggctttcacccgcaggtcactaaacaggttgacccgagaaaactgaccgacagaagcggaaaataa

SEQ ID NO: 2 - protein, 383 aa, Bacillus Subtilis strain ARRMK-33
MNHSKTLLLTAAAGLMLTCGAVSSQAKHKLSDPYHFTVNAAAETEPVDTAGDAADDPAIWLDPKNPQNSKLITTNKKSGLVVYSLEGKMLHSYPTGKLNNVDIRYDFPLNGKKVDIAAASNRSEGKNTIEIYAIDGKNGTLQSITDPDRPIASAIDEVYGFSLYHSQKTGKYYAMVTGKEGEFEQYELNADKNGYISGKKVRAFKMNSQTEGMAADDEYGSLYIAEEDEAIWKFSAEPDGGSNGTVIDRADGRHLTPDIEGLTIYYAADGKGYLLASSQGNSSYAIYERQGQNKYVADFQITDGPETDGTSDTDGIDVLGFGLGPEYPFGFFVAQDGENIDHGQKANQNFKMVPWERIADKIGFHPQVTKQVDPRKLTDRSGK

SEQ ID NO: 3 - protein, 442 aa, Aspergillus fumigatus
SAGSKSCDTVDLGYQCSPATSHLWGQYSPFFSLEDELSVSSKLPKDCRITLVQVLSRHGARYPTSSKSKKYKKLVTAIQANATDFKGKFAFLKTYNYTLGADDLTPFGEQQLVNSGIKFYQRYKALARSVVPFIRASGSDRVIASGEKFIEGFQQAKLADPGATNRAAPAISVIIPESETFNNTLDHGVCTKFEASQLGDEVAANFTALFAPDIRARAEKHLPGVTLTDEDVVSLMDMCSFDTVARTSDASQLSPFCQLFTHNEWKKYNYLQSLGKYYGYGAGNPLGPAQGIGFTNELIARLTRSPVQDHTSTNSTLVSNPATFPLNATMYVDFSHDNSMVSIFFALGLYNGTEPLSRTSVESAKELDGYSASWVVPFGARAYFETMQCKSEKEPLVRALINDRVVPLHGCDVDKLGRCKLNDFVKGLSWARSGGNWGECFS

SEQ ID NO: 4 - protein, 467 aa, Aspergillus niger
MGVSAVLLPLYLLSGVTSGLAVPASRNQSTCDTVDQGYQCFSETSHLWGQYAPFFSLANESAISPDVPAGCKVTFAQVLSRHGARYPTDSKGKKYSALIEEIQQNATTFDGKYAFLKTYNYSLGADDLTPFGEQELVNSGIKFYQRYESLTRNIIPFIRSSGSSRVIASGKKFIEGFQSTKLKDPRAQPSQSSPKIDVVISEASSSNNTLDPGTCAVFEDSELADTVEANFTATFVPSIRQRLGNDLSGVSLTDTEVTYLMDMCSFDTISTSTVDTKLSPFCDLFTHDEWINYDYLQSLKKYYGHGAGNPLGPTQGVGYANELIARLTHSPVHDDTSSNHTLDSSPATFPLNSTLYADFSHDNGIISILFALGLYNGTKPLSTTTVQNITQTDGFSSAWTVPFASRLYVEMMQCQAEQEPLVRVLVNDRVVPLHGCPADALGRCTRDSFVRGLSFARSGGDWAECFA

SEQ ID NO: 5 - protein, 448 aa, Aspergillus oryzae
LAVPASRNQSTCDTVDQGYQCFSETSHLWGQYAPFFSLANKSAISPDVPAGCHVTFAQVLSRHGARYPTDSKGKKYSALIEEIQQNATTFEGKYAFLKTYNYSLGADDLTPFGEQELVNSGVKFYQRYESLTRNIVPFIRSSGSSRVIASGNKFIEGFQSTKLKDPRAQPGQSSPKIDVVISEASTSNNTLDPGTCTVFEDSELADDIEANFTATFVPSIRQRLENDLSGVSLTDTEVTYLMDMCSFDTISTSTVDTKLSPFCDLFTHEEWINYDYLQSLNKYYGHGAGNPLGPTQGVGYANELIARLTHSPVHDDTSSNHTLDSNPATFPLNSTLYADFSHDNGIISILFALGLYNGTKPLSSTTAENITQTDGFSSAWTVPFASRMYVEMMQCQSEQEPLVRVLVNDRVVPLHGCPVDALGRCTRDSFVKGLSFARSGGDWGECFA

SEQ ID NO: 6 - protein, 421 aa, Raoutella sp.
MPARVQGLLRLFIACALPLLALHSAAAERYQLEKVVELSRHGIRPPTAGNNEEIIAATGRPWTEWTTHDGELTGHGYAAVVNKGREEGQHYRQLGLLQAGCPTAESIYVRASPLQRTRATAQALVDGAFPGCGVAIHYVNGDADPLFQTDKFAATQTDPARQLAAVKEKAGDLAQRRQALEPTIQLLKVAVCQADKPCPIFDTPWQVEQSKSGKTTISGLSVMANMVETLRLGWSENLPLSQLAWGKIAQASQITALLPLLTENYDLSNDVLYTAQKRGSILLNAMLQGVEEGARPDVRWLLLVAHDTNIAMVRTLMNFSWQLPGYSRGNIPPGSSLVLERWRDAKSGRYFLRVDFQAQGLDDLRRLQTPDAQHPMLRQEWRQPGCRQTDVGTLCPFQAAITALGQRIDRSSTPAVAMVLP

SEQ ID NO: 7 - protein, 432 aa, E.coli
MKAILIPFLSLLIPLTPQSAFAQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 8 - protein, 270 aa, Enterobacter
MMKTSAKLAASGLVALLLTGCASSTHQTAQQQLGQQSVLAVNWFQQSGEYQALTWQAFNTARMAFDQAPSLTGKPKAVIVDLDETMLDNSAYSAWQAKNGQPFSSKTWSAWTQARQAKAVPGAVEFARHVTENGGTLFYVSNRDQKDYAATVANMQQLGFPNVSDKTVRLNTDSSNKQARFDAIKNAGYNVVLYVGDNLNDFGGATWHKGNQTRRDFVNLNHQQFGTQFIVLPNPLYGDWESGMAENYNKLTPEQQLSVRESRLQSWNGK

SEQ ID NO: 9 - protein, 383 aa, Bacillus subtilis
MNHSKTLLLTAAAGLMLTCGAVSSQAKHKLSDPYHFTVNAAAETEPVDTAGDAADDPAIWLDPKTPQNSKLITTNKKSGLVVYSLDGKMLHSYNTGKLNNVDIRYDFPLNGKKVDIAAASNRSEGKNTIEIYAIDGKNGTLQSMTDPDHPIATAINEVYGFTLYHSQKTGKYYAMVTGKEGEFEQYELKADKNGYISGKKVRAFKMNSQTEGMAADDEYGRLYIAEEDEAIWKFSAEPDGGSNGTVIDRADGRHLTRDIEGLTIYYAADGKGYLMASSQGNSSYAIYDRQGKNKYVADFRITDGPETDGTSDTDGIDVLGFGLGPEYPFGIFVAQDGENIDHGQKANQNFKIVPWERIADQIGFRPLANEQVDPRKLTDRSGK

SEQ ID NO: 10 - protein, 383 aa, Bacillus amyloliquefaciens
MNHSKTLLLTAAAGLMLTCGAVSSQGKHKLSDPYHFTVNAAAETEPVDTAGDAADDPAIWLAPKNPQNSKLITTNKKSGLVVYSLEGKTLHSYHTGKLNNVDIRYDFPLNGKKVDIAAASNRSEGKNTIEIYAIDGKNGTLQSITDPDRPIASAIDEVYGFSLYHSQKTGKYYAMVTGKEGEFEQYELNADKNGYISGKKVRAFKMNSQTEGMAADDEYGSLYIAEEDEAIWKFSAEPDGGSNGTVIDRADGRHLTPDIEGLTIYYAADGKGYLLASSQGNSSYAIYERQGQNKYVADFQITDGPETDGTSDTDGIDVLGFGLGPEYPFGLFVAQDGENIDHGQKVNQNFKMVPWERIADKIGFHPQVNKQVDPRKLTDRSGK

SEQ ID NO: 11 - protein, 375 aa, Bacillus licheniformis
MNFYKTLALSTLAASLLSPSWSILPRAEASAYKDFSVTADAETEPVDTPDDAADDPAIWVHPKQPEKSRLITTNKKSGLIVYDLNGKQLAAYPFGKLNNVDLRYNFPLDGKKIDIAGASNRSDGKNTVEIYAFDGEKNKLKNIVNPQKPIQTDIEEVYGFSLYHSQKTGKFYAMVTGKNGEFEQYELFDNGKGQVEGKKVRSFKMSSQTEGLAADDEYGKMYIAEEDAAIWSFSAEPNGGDKGKIVDRAGGPHLTADIEGLTIYYGEDGEGYLIASSQGDNRYAIYDRRGKNDYVADFSIDDGKEIDGTSDTDGIDVIGFGLGKKYPYGIFVAQDAKIRKMDSQPIRTSKLSPGKKLLTRWTTSLISMIRSIPEN

SEQ ID NO: 12 - DNA, 35 bp, artificial sequence
  chemically synthetized
aattcatgaatcattcaaaaacacttttgttaacc

SEQ ID NO: 13 - DNA, 30 bp, artificial sequence
  chemically synthetized
tacgtcgacttattttccgcttctgtcggt

(19) United States
(12) Patent Application Publication
(10) Pub. No.: US 2009/0047707 A1
(43) Pub. Date: Feb. 19, 2009

(54) Bacterial recombinant phytase

(75) Inventors: Ramachandra Reddy Arjula, Hyderabad (IN); Kodandarami Reddy Malireddy, New Delhi (IN); Ramakrishnareddy Isanaka, Warren, NJ (US)

Correspondence Address: John Dodds, 1707 N St. NW, Washington, DC, 20036, US

(73) Assignee: Issar Pharmaceuticals Pvt Ltd, Hyderabad (IN)

(21) Appl. No.: 11/893,942

(22) Filed: Aug. 17, 2007

(51) Int. Cl. C12P 21/04; C12N 1/20; C12N 15/00; C12N 15/11; C12N 9/14

(52) U.S. Cl. 435/69.1; 435/195; 435/252.3; 435/252.33; 435/320.1; 536/23.2

(57) ABSTRACT

A novel phytase enzyme obtainable from B. Subtilis strain ARRMK-33 is disclosed. Also a novel method to produce recombinant phytase protein in prokaryotic cells is disclosed.

### BACKGROUND OF THE INVENTION

1. Field of the Invention

The present invention relates to a novel bacterial phytase, nucleic acid sequence coding for the same, a recombinant vector containing the nucleic acid sequence, method to produce the phytase and uses thereof.

2. Description of the Related Art

During the last 20 years, phytases have attracted considerable attention from both scientists and entrepreneurs in the areas of nutrition, environmental protection, and biotechnology. These enzymes belong to a special class of phosphomonoesterases [myo-inositol hexakisphosphate 3-phosphorylase (EC 3.1.3.8) and myo-inositol hexakisphosphate 6-phosphorylase (EC 3.1.3.26)] and are capable of initiating the stepwise release of phosphate from phytate [myo-inositol (1, 2, 3, 4, 5, 6) hexakisphosphate], the major storage form of phosphate in plant seeds and pollen (Greiner et al., 2002). Phytases were originally proposed as an animal feed additive to enhance the nutritional quality of plant material in feed for simple-stomached animals by liberating phosphate (Miksch et al., 2002). More recently, addition of phytase enzyme has been observed as a means to reduce the level of phosphate pollution in areas of intensive animal production such as poultry or dairy production. Numerous studies have shown the effectiveness of supplemental phytases of microbial origin in improving utilization of phosphate from phytate containing diet (Cromwell et al., 1995, Igbasan et al., 2001, Leesen et al., 2000, Simons et al., 1990, Walz. et al., 2002). Thus, the inorganic phosphate supplementation in the diets for simple-stomached animals can be substantially reduced by including adequate amounts of phytase, and as a result, the faecal phosphate excretion of these animals may be reduced by as much as 50%. Because phytate acts as an anti-nutrient by binding to proteins and by chelating minerals (Cheriyan 1980, Reddy et al., 1989), addition of phytase can improve the nutritional value of plant-based foods by enhancing protein digestibility and mineral availability. This is mainly through phytate hydrolysis during digestion in the stomach or during food processing (Sandberg et al., 2002).

Since certain myo-inositol phosphates have been proposed to have novel metabolic effects (Ohkawa et al., 1984, Potter, 1995, Shamsuddin, 2002, Vucenik et al., 2003), phytases may also find application in food processing to produce functional foods (Konietzny et al., 2003). Phytases have a wide distribution in plants, microorganisms, and also in some animal tissues (Vohra et al., 2003). Recent research has shown that microbial phytases are the most promising ones for biotechnological application in terms of cost, ease of production and processing (Pandey et al., 2001). Although phytases from several species of bacteria, yeast and fungi have been characterized commercial production currently focuses mainly on the soil fungus Aspergillus. However, due to some properties, such as substrate specificity, resistance to proteolysis and catalytic efficiency, bacterial phytases emerged as a real alternative to the fungal enzymes. Phytases have been detected in various bacteria, such as Pseudomonas sp. (Irwing et al., 1971, Richardson et al., 1997), Bacillus sp. (Choi et al., 2001, Kerovuo et al., 1998), Raoultella sp. (Sajidan et al., 2004, Shah et al., 1990), Escherichia coli (Greiner et al., 1993), Citrobacter braakii (Kim et al., 2003), Enterobacter (Yoon et al., 1996) and anaerobic rumen bacteria, particularly in Selenomonas ruminantium, Megasphaera elsdenii, Prevotella sp., Mitsuokella multiacidus (Yanke et al., 1998), and Mitsuokella jalaludinii (Lan et al., 2002). With lactic acid bacteria, however, the results were inconsistent; a few strains seem to have a quite low phytase activity, but with the majority of strains the detection of significant phytase activity failed. Recently it was shown that lactic acid bacteria isolated from sourdoughs exhibit a considerable phytate degrading capacity (Angelis et al., 2003). Among the different lactic acid bacterial strains isolated from sourdoughs, Lactobacillus sanfranciscensis, which is considered as a key sourdough lactic acid bacterium, was identified as the best phytase producer.

Phytase has also been detected in various bacteria, e.g. Aerobacter aerogenes (Greaves et al., 1967), Pseudomonas sp. (Irving and Cosgrove, 1971), Bacillus subtilis (Powar and Jagannathan, 1982), Klebsiella sp. (Shah and Parekh, 1990), B. subtilis (Shimizu, 1992), Escherichia coli (Greiner et al., 1993), Enterobacter sp. 4 (Yoon et al., 1996) and Bacillus sp. DS11 (later designated as B. amyloliquefaciens) (Kim et al. 1998a). Generally, the phytases produced by fungi are extracellular, whereas the enzymes from bacteria are mostly cell associated. The only bacteria showing extracellular phytase activity are those of the genera Bacillus and Enterobacter. The phytases of Escherichia coli have been reported to be periplasmatic enzymes and phytase activity in Selenomonas ruminantium and Mitsuokella multiacidus was found to be associated with the outer membrane (D'Silva et al., 2000).

Apart from fungi and bacteria, phytase has been isolated and characterized from cereals such as triticale, wheat, maize, barley and rice and from beans such as navy beans, mung beans, dwarf beans and California small white beans (Reddy et al., 1993).

Phytases are of great interest for biotechnological applications, in particular for the reduction of phytate content in feed and food (Lei et al., 2001, Vohra et al., 2003). Depending on the application, a phytase in which there is commercial interest should fulfill a series of quality criteria. Enzymes used as feed additives should be effective in releasing phytate phosphate in the digestive tract, stable to resist inactivation by heat from feed processing and storage, and cheap to produce. Thermostability is a particularly important issue since feed pelleting is commonly performed at temperatures between 65° C. and 95° C. Although phytase inclusion using an after-spray apparatus for pelleted diets and/or chemical coating of phytase may help bypass or overcome the heat destruction of the enzyme, thermostable phytases will no doubt be better candidates for feed supplements.

Naturally occurring phytases having the required level of thermostability for application in animal feeding have not been found in nature thus far (Lei et al., 2001). Up to now, two main types of phytases have been identified; acid phytases with a pH optimum around pH 5.0 and alkaline phytases with a pH optimum around pH 8.0 (Konietzny et al., 2002). Most of the so far described microbial phytases belong to the acidic ones and their pH optima range from 4.0 to 5.5.

Finally, a phytase will not be competitive if it cannot be produced in high yield and purity by a relatively inexpensive system. Recently, economically competitive expression and/or secretion systems for microorganisms have been developed. A different strategy to overcome the problems using phytases as a feed additive such as cost, inactivation at the high temperatures required for pelleting feed, and loss of activity during storage, might be to add those enzymes to the repertoire of digestive enzymes produced endogenously by swine and poultry. The food industry may also be interested in using phytases; on the one hand to improve mineral bio-availability by reducing phytate content of a given food, on the other hand to produce functional foods. Certain myo-inositol phosphates have been suggested to have beneficial health effects, such as reducing the risk of heart disease, renal stone formation, and certain type of cancers. The number and position of the phosphate groups on the myo-inositol ring is thereby of great significance for their physiological functions.

### SUMMARY OF THE INVENTION

Due to the increased interest in phytases, especially in the area of food and feed production, but also in other areas, there is a clear need for novel phystase enzymes. Especially there is a need for novel phytase enzymes possessing high specific activity, thermostablility, and activity in a broad range of pH. Moreover, there is a need for a method for economic production of large quantities of phytase fulfilling the above criteria.

Accordingly, an object of the current invention is to provide a novel recombinant phytase which is heat stable and active in a broad rage of pH and which can stand food pelleting processes and conditions.

Another object of the current invention is to provide a simple and rapid method of induction of recombinant phytase expression in host cell and subsequent purification of recombinant phytase that has a low Km for phytate, thereby bringing down the cost as the specific activity is rather high.

An even further object of the current invention is to provide a novel phytase that can be used in human diet to improve digestion of phytate.

A further object of the current invention is to provide a novel thermostable phytase to be used for removal of plant phytic acid during pulp and paper processing.

The phytase of the present invention is obtainable from Bacillus subtilis strain ARRMK 33 which is deposited at American type Culture Collection, Manassas, Va.,

Other objects of the present invention will become apparent from the following detailed specification.

A preferred embodiment, phytase enzyme of the present invention comprises the amino acid sequence essentially according to SEQ ID NO: 2.

Another preferred embodiment of the current invention is a method to produce recombinant phytase having an amino acid sequence essentially according to SEQ ID NO: 2, and further being thermostable up to about 70° C., and having optimum pH range between 5.5 and 7.5.

An even flurther embodiment of the current invention is a cloning strategy of a novel phytase gene obtainable from B. subtilis strain ARRMK-33 (Deposited with American Type Culture Collection), where the DNA sequence essentially according to SEQ ID NO:2 is cloned into TOPO vector and PCR cloned into H-vector.

Yet another embodiment of the current invention is an expression vector carrying strong leftward promoter pL of coliphage A and c1857 allele coding for a thermolabile repressor protein and the phytase gene obtainable from B. subtilis straing ARRMK-33 downstream from the pL promoter.

A further embodiment of the current invention is a transformation vector pTF16 containing the expression vector carrying the recombinant phytase encoding gene, pL promoter and c1857 allele.

Still another embodiment of the current invention is a method to produce recombinant phytase having amino acid sequence essentially according to SEQ ID NO:2, optimum pH between 5.5 and 7.5. And said recombinant phytase being thermostable up to 70° C., said method comprising the steps of a) transforming E.coli cells with transformation vector pTF16 containing expression vector carrying the recombinant phytase encoding gene, pL promoter and c1857 allele, b) cultivating the E. coli cells after increasing culture temperature to 42° C. degrees in order to denature the c1857 repressor protein and receive strong expression of recombinant phytase protein, d) centrifuging the medium and e) collecting the recombinant phytase from the soluble fraction.

### BRIEF DESCRIPTION OF THE DRAWINGS

FIG. 1 depicts a cloning strategy of the phytase enzyme

FIG. 2. depicts a flow chart of assay for determination of phytase activity in bacterial crude cultures

FIG. 3. depicts the DNA sequence (SEQ ID NO:1) of the phytase clone.

FIG. 4. Depicts the amino acid sequence (SEQ ID NO: 2) of the recombinant phytase

FIG. 5. Depicts the amino acid sequence of phytase of Aspergillus fumigatus (SEQ ID NO:3), Aspergillus niger (SEQ ID NO:4), Aspergillus oryzae (SEQ ID NO:5), Raoultella sp. (SEQ ID NO:6), Escherichia coli (SEQ ID NO:7), Enterobacter (SEQ ID NO:8), Bacillus subitlis (SEQ ID NO:9), Bacillus amyloliquefaciens (SEQ ID NO:10), and Bacillus licheniformis (SEQ ID NO:11)

FIG. 6 shows multiple alignment of ARRMK-33 sequence (SEQ ID NO:2) with other phytase sequences: Aspergillus fumigatus (SEQ ID NO:3), Aspergillus niger (SEQ ID NO:4), Aspergillus oryzae (SEQ ID NO:5), Raoultella sp. (SEQ ID NO:6), Escherichia coli (SEQ ID NO:7), Enterobacter (SEQ ID NO:8), Bacillus subitlis (SEQ ID NO:9), Bacillus amyloliquefaciens (SEQ ID NO:10), and Bacillus licheniformis (SEQ ID NO:11).

FIG. 7. Depicts a dendrogram showing the evolutionary relationship of ARRMK phytase with other related sequences

FIG. 8. Depicts TOPO cloning vector

FIG. 9. Depicts PL 452 Coliphage harboring H-vector

FIG. 10. Depicts H-vector harboring phytase encoding sequence

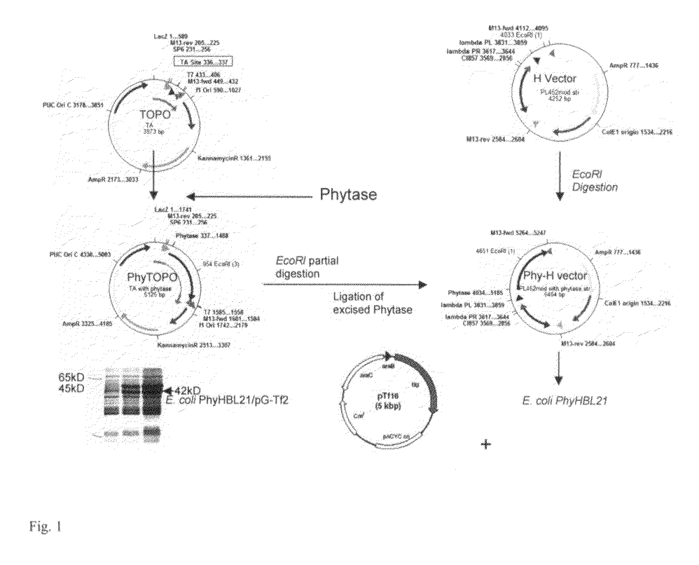

FIG. 1 1. Depicts chaperone vector pTf16

FIG. 12. Depicts expression profile of recombinant phytase expression vector in pTf16 in chaperone vector

FIG. 13 Depicts phosphate standard graph

FIG. 14. Depicts effect of temperature of phytase activity

FIG. 15. Depicts effect of pH on phytase activity

### DETAILED DESCRIPTION OF THE INVENTION

The present invention relates to isolation and cloning of a novel DNA sequence from a bacterial strain (Bacillus subtilis ARRMK33) encoding phytase (myo-inositol hexakisphophate phosphodydrolyase) enzyme. Phytase catalyses hydrolysis of myo-inositol hexakisphosphate to inorganic phosphate and lowers myo-inositol phosphates and, in some cases even myo-inositol. The coding sequence of the novel phytase of this disclosure was inserted into a plasmid construct, which in turn was inserted into a plasmid expression vector capable of effectively transforming a microbial expression host. A cost effective system is disclosed for optimal expression of this transgene and purification of the gene product is disclosed. This embodiment of the recombinant gene sequence may be used to economically produce phytase on an industrial scale at comparably lower cost. The phytase produced through this novel process may be used in a variety of processes requiring conversion of phytate to inositol and inorganic phosphate. These include poultry, dairy and also human food. The novel recombinant phytase is heat stable, active in broad range of pH and can withstand food pelleting processes and conditions. Also the present invention deals with a simple and rapid method of induction and purification of phytase that has a low Km for phytate, therefore bringing down the costs as the specific activity is relatively high.

According to the present disclosure a novel phytase DNA sequence was isolated and cloned from Bacillus subtilis ARRMK33 the DNA encodes for a novel phytase enzyme essentially having amino acid sequence according to SEQ ID NO:2.

According to the present disclosure the coding sequence of the novel phytase is expressed in an expression vector that carries a strong leftward promoter pL of coliphage A as well as c1857 allele coding for a thermolabile repressor protein. The coding region of the novel phytase gene was cloned downstream from the pL promoter. The recombinant plasmid was introduced to E coli strain and coexpressed with a chaperonin vector. Heat inducible synthesis of the novel recombinant phytase is obtained by increasing the culture temperature to 42° C. Novel recombinant phytase enzyme is purified from the soluble portion of the culture.

The invention is more closely illustrated by the following examples, which are not meant to limit the scope of the invention.

### EXAMPLE 1

### Screening of Bacillus Strains for Phytase Production

Around 50 samples were collected from different soils and the Bacillus cultures were tested for extracellular phytase production in Luria broth supplemented with phytate, and in wheat bran extract medium. None of the strains produced phytase activity in the Luria broth, whether or not it was supplemented with phytate. However, in the wheat bran medium, five B. subtilis strains produced significant amounts of phytase activity. The B. subtilis strain ARRMK-33 showed the highest phytase activity and was therefore chosen for isolation of phytase gene and phytase enzyme production in a heterologous system. The strain is deposited at American Type Culture Collection.

### EXAMPLE 2

### Phylogenetic Analysis of the Novel Phytase

Homologous sequences of the novel phytase were obtained from public domain databases with BLASTP program and conserved regions were analyzed

Multiple alignment (FIG. 6) of highly homologous sequences and phylogenetic analysis shows that ARRMK-33 is close to Bacillus subtilis and B. amyloliquefaciens. A dendrogram showing the evolutionary relationship of tARRMK-33 phytase with other related sequences is shown in FIG. 7.

### EXAMPLE 3

### Expression of Recombinant Phytase in E. coli

Bacillus subtilis (ARRMK-33) DNA was isolated from single colony cultures and amplified with gene specific primers of phytase (5′ AAT TCA TGA ATC ATT CAA AAA CAC TTT TGT TAA CC 3′ (SEQ ID NO: 12) and 5′ TAC GTC GAC TTA TTT TCC GCT TCT GTC GGT 3′(SEQ ID NO:13)) containing SalI restriction site in reverse primer. EcoRI adapters were ligated to PCR amplicons and digested with SalI to create cohesive ends with EcoRI in 5′ region and SalI site in the 3′ of the amplified product The PCR product was ligated with EcoRI and SalI digested heat inducible vector at 16° C. The positive colonies were screened with colony PCR and plasmid DNA was isolated. This construct was mobilized into E. coli strain BL21 (DE3). E.coli (BL21) containing the expression vector carrying the phytase gene was grown overnight in LB medium at 28° C. with ampicillin as selectable marker until OD reaches ˜0.6. The temperature of the incubator was raised to 42° C. as to release the repressor and to induce recombinant phytase gene expression. The rPhytase protein was included in inclusion bodies and collected in pellet. In order to collect the rPhytase in soluble fraction, it was co-expressed with pTf-16 (FIG. 11) and with the chaperonin activity rPhytase was collected in the soluble fraction. The cloning strategy is illustrated in FIG. 1.

### EXAMPLE 4

### Bacterial Transformation

The E. coli strain DH5α was grown at 37° C. either on solid (1.5% agar) or in liquid LB medium (1% tryptone, 1% NaCl, 0.5% yeast extract). Liquid cultures were grown initially in 2 ml of LB medium in a test tube, and later in 1-liter flasks for plasmid isolation.

Competent cells of E. coli were prepared as follows. One ml of DH5α cells from an overnight grown culture was inoculated in 100 ml of LB medium without antibiotic. The cells were grown till they reached an A₆₀₀ of 0.4-0.6. Cells were then harvested into precooled 50 ml falcon tubes by centrifugation at 3000 rpm for 10 min at 4° C. All the operations were performed under sterile conditions at 4° C. After the centrifugation, the cells were re-suspended in 15 ml 0.1 M CaCl₂ and incubated on ice for 10 min. This suspension was centrifuged at 3000 rpm for 10 min. The resultant pellet was re-suspended in 4 ml of 0.1 M CaCl₂ (in 10% glycerol) for every 100 ml of original culture, dispensed into 200 μl aliquots, frozen and stored at −70° C. for future use.

Transformation of the competent cells was done as follows: Frozen E. coli cells were thawed on ice to which 1 ng of plasmid DNA or 100 ng of ligation mix were added. The suspension was carefully mixed with pipette tip and incubated on ice for 30 min. A heat shock of 42° C. for 45 sec was applied followed by incubation on ice for another 2 min. 800 μl of LB was added and the bacterial suspension was incubated at 37° C. with shaking for 1 h. Aliquots of the suspension were spread evenly on LB supplemented with an appropriate antibiotic. The plates were incubated at 37° C. overnight. Following day, single colonies were picked up and inoculated for plasmid mini preparation.

### EXAMPLE 5

### Plasmid DNA Isolation

A single colony of the E. coli strain DH5α, carrying the plasmid of interest, was inoculated into 5 ml of LB medium containing the appropriate antibiotic and incubated overnight with shaking at 37° C. An aliquot of 1.5 ml of the culture was transferred to a 1.7 ml tube and spun in a microcentrifuge for 1 min at 14,000 rpm at 4° C. The supernatant was removed by aspiration. The pellet was suspended in 100 μl of GTE solution (50 mM glucose, 25 mM Tris.Cl pH 8.0, 10 mM EDTA pH 8.0) by vortexing. Then 200 μl of freshly prepared lysis solution (0.2 N NaOH, 1% SDS) was added and the contents were mixed and stored at room temperature for 5 min. Then the solution was neutralized by 150 μl of 3 M potassium acetate pH 4.8, mixed by inversion and stored on ice for 10 min. The cellular debris was removed by centrifugation at 14,000 rpm for 10 min at 4° C. The supernatant was transferred to a fresh tube and DNase free RNase was added at a final concentration of 20 μg/ml and incubated at 37° C. for 20 min. After the RNase treatment, the suspension was extracted twice with phenol:chloroform (1:1) and once with chloroform. Then the plasmid DNA in the aqueous phase was precipitated with 0.6 volume of isopropanol. The DNA pellet was washed with 70% ethanol, dried, dissolved in TE and stored at −20° C.

### EXAMPLE 6

### Prokaryotic Over Expression Strategy

We have constructed an expression vector that carries the strong leftward promoter (pL) of coliphage A as well as the ce1857 allele, which codes for a thermolabile repressor protein. The coding region of the novel Bacillus phytase gene was cloned downstream from the pL promoter. The recombinant plasmid was introduced into BL21 Escherichia coli strain. Heat-inducible synthesis of the novel Bacillus phytase was obtained, when the culture temperature increased from 30° C. to 42° C., c1857 repressor protein denatured and the pL promoter is activated strongly leading to the overproduction of the recombinant phytase protein.

### EXAMPLE 7

### Construction of the Phytase Expression Vector

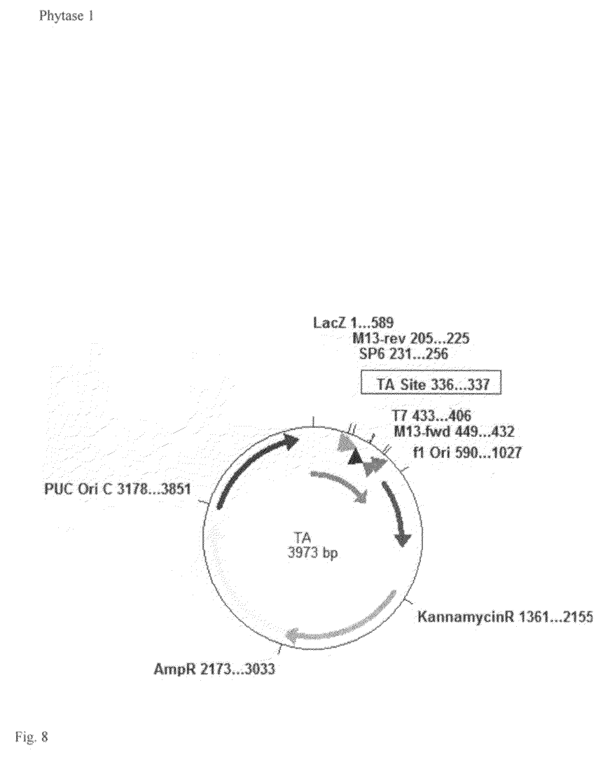

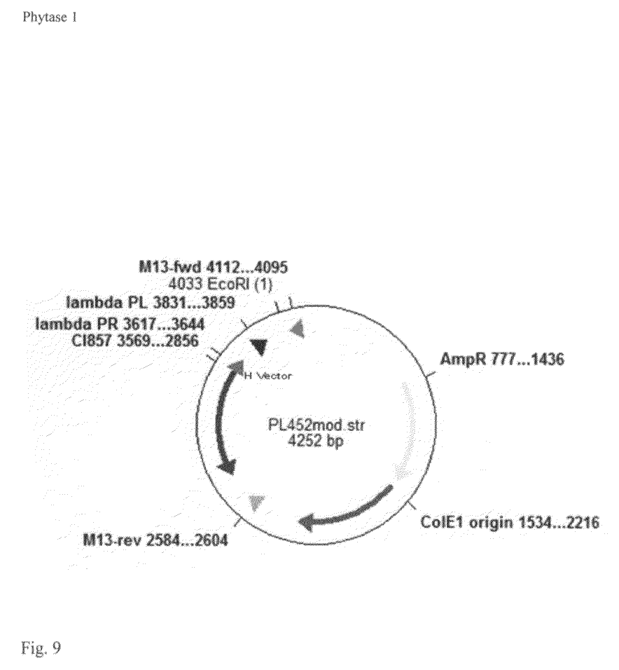

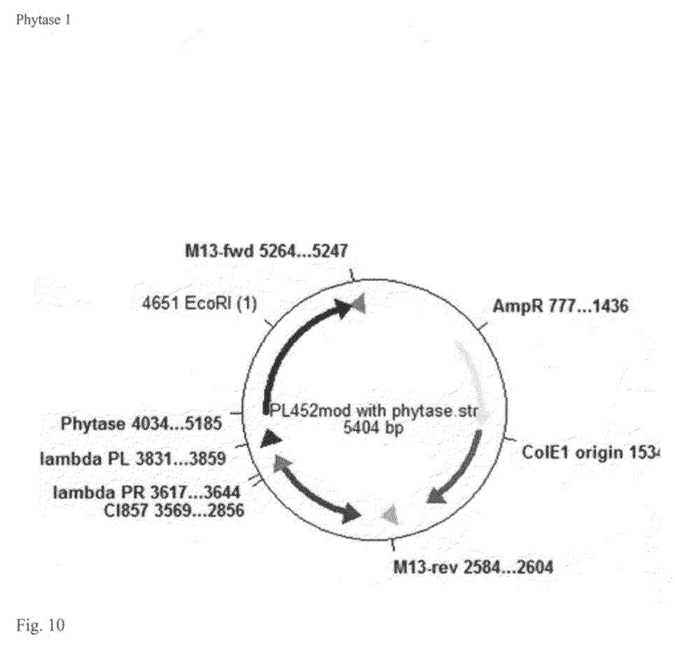

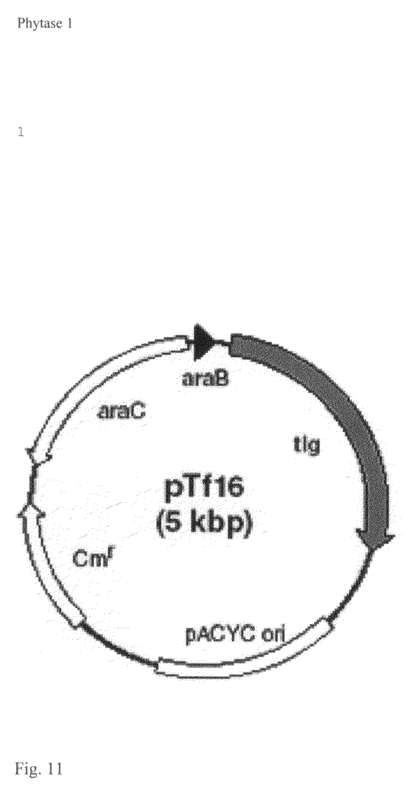

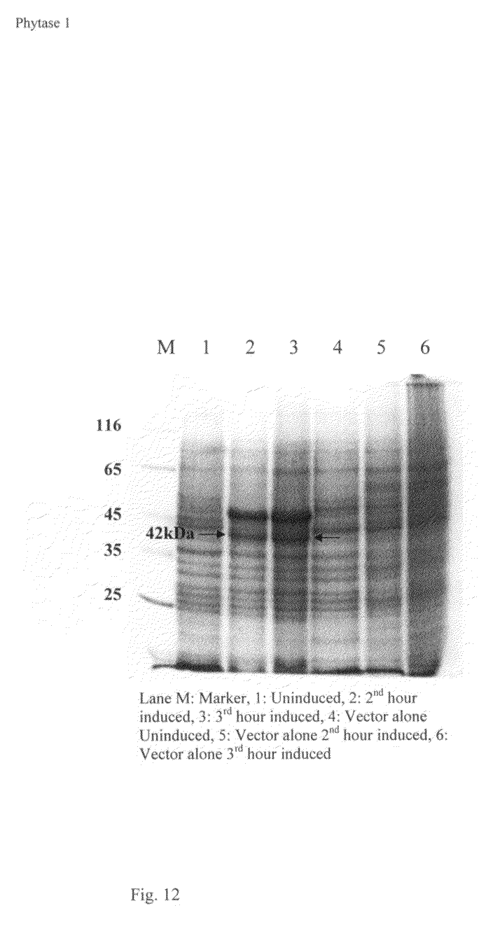

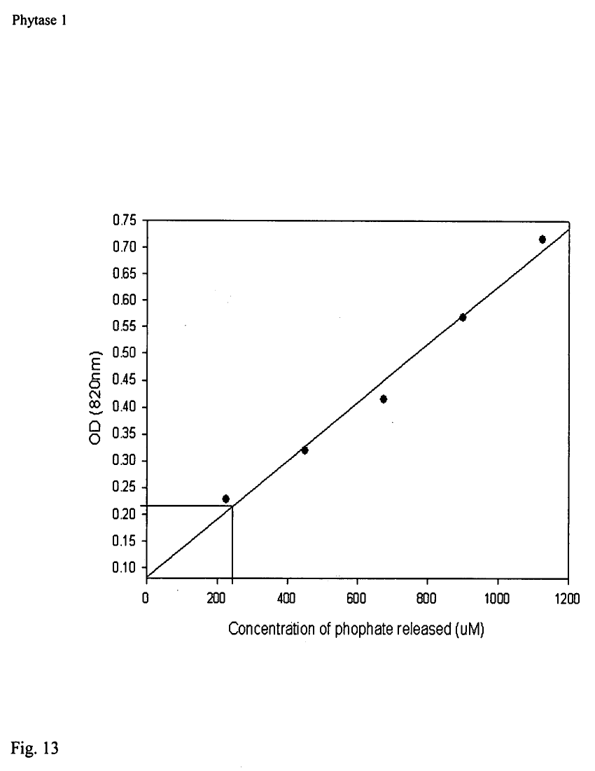

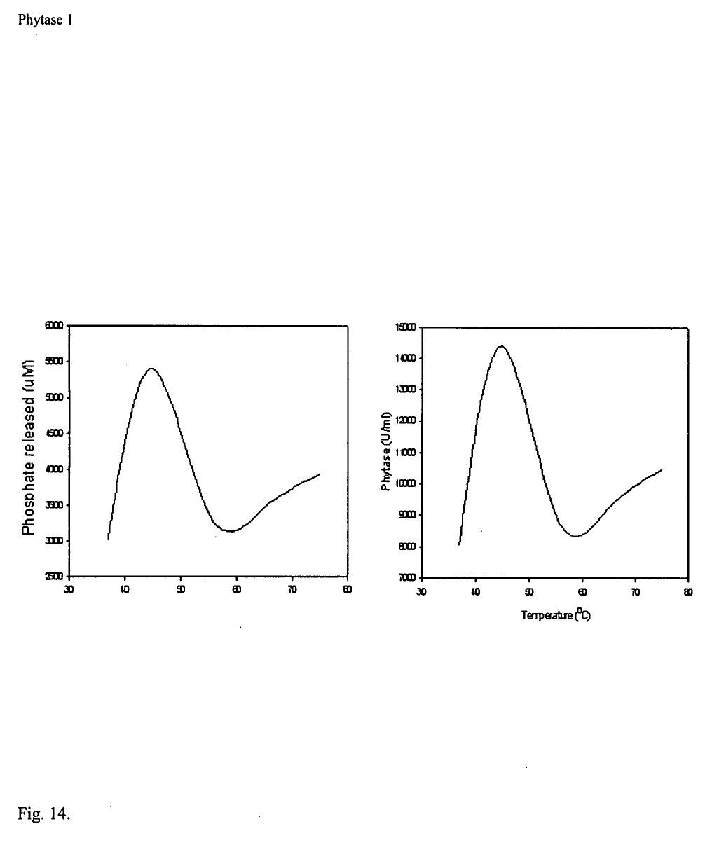

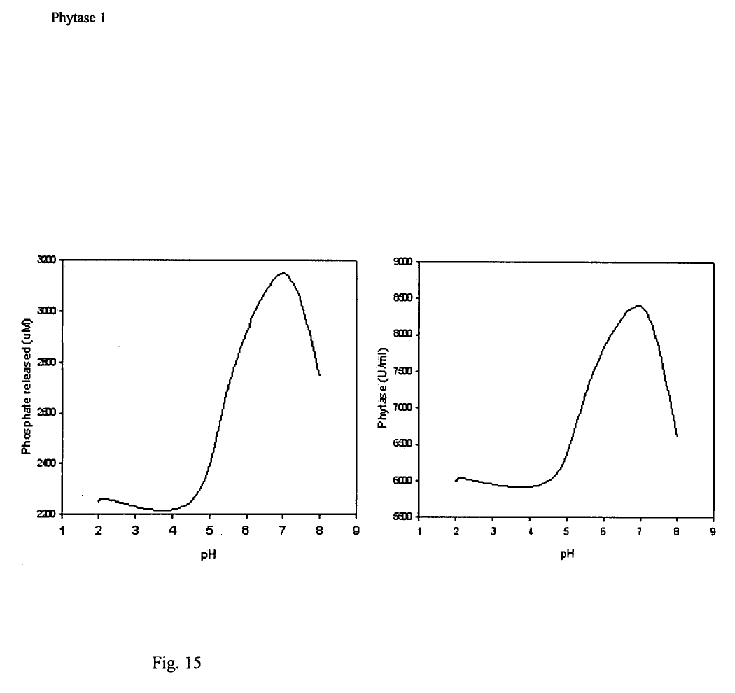

The 1.1 kb phytase gene was amplified using gene specific primers and cloned into TA cloning vector (FIG. 8). Transformation was done using DH5α competent cells and plated on amp⁺ resistant plates. The positive colonies were screened for phytase gene through colony PCR and resolved on 1% agarose gel. The 1.1 kb fragment was excised from TA vector through partial digestion with EcoRI and was cloned into the same site of H-vector (FIG. 9). The recombinant plasmid was transformed into E. coli strain BL21 (DE3). The positive colonies of Phytase in E. coli strain BL21 (DE3) screened through colony PCR. The positive clones harboring recombinant phytase in H-vector (FIG. 10) was co transformed with pTF16 chaperone vectors (FIG. 11).

### EXAMPLE 8

### Growth of E. coli, Induction of rPhytase Production and Purification of the Recombinant Phytase Enzyme

Transformation vector pTF16 containing the expression vector carrying the rPhytase was grown in LB medium at 37° C. in an orbital shaker until the A₆₀₀ of the culture reached 0.6-0.9. At this stage the cells were induced at 42° C. and the cultures were allowed to grow for an additional three hours. Samples were collected at 1 hr intervals for three hours and were analyzed on 10% SDS PAGE gels. Cells were harvested by centrifugation at 5000 rpm, 15 min at room temperature. Recombinant phytase was collected from the soluble fraction.

SDS-PAGE was performed according to Laemmli et al., (1970). About 20 μg of crude protein was loaded on mini gels. The separation and stacking gel composition is as follows: Separating gel solution (15 ml) contains 5 ml of 30% acrylamide solution, 3.8 ml of 1.5M Tris.Cl pH 8.8, 150 μl of 10% SDS, and 5.9 ml of distilled water, 150 μl of 10% ammonium persulphate (APS), and 6 μl of TEMED. Stacking gel solution (5 ml) contains 0.83 ml acrylamide (30%), 630 μl of 1M Tris.Cl pH 6.7, 50 μl of 10% SDS, 3.4 ml of water, 50 μl of 10% APS, and 5 μl of TEMED. Electrophoresis was carried out at 150V after which the gels were stained with Comassie Blue. Gels were destained with a solution containing 7.5% methanol and 7% glacial acetic acid. From these gels the induced rPhytase protein was identified by comparison with that of the uninduced sample (FIG. 12)

### EXAMPLE 9

### Characterization of the Recombinant Phytase Enzyme

### Phytase Activity Assay:

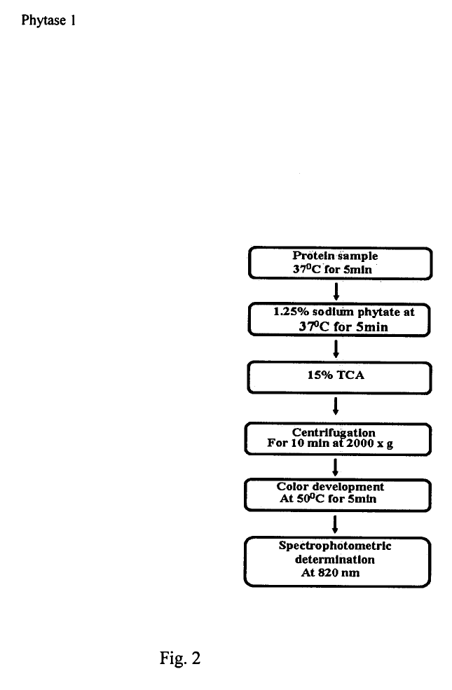

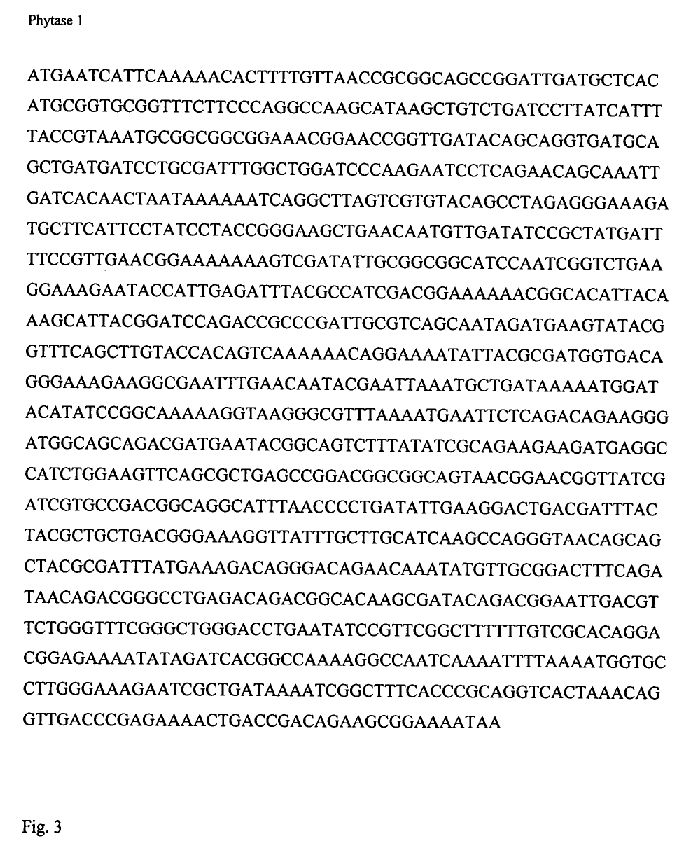

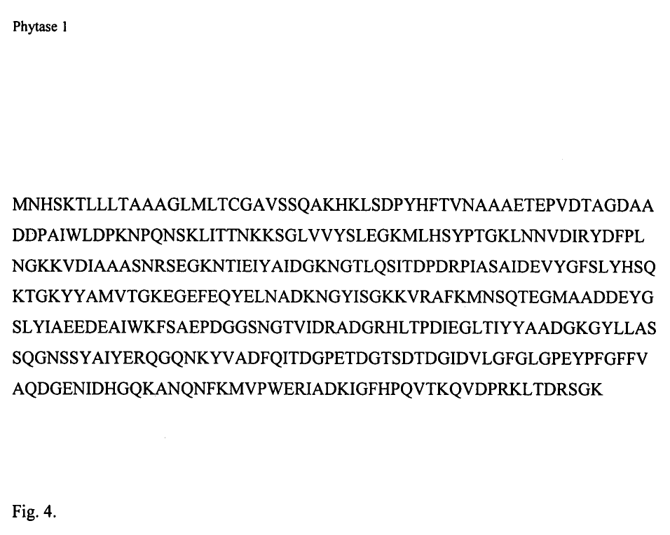

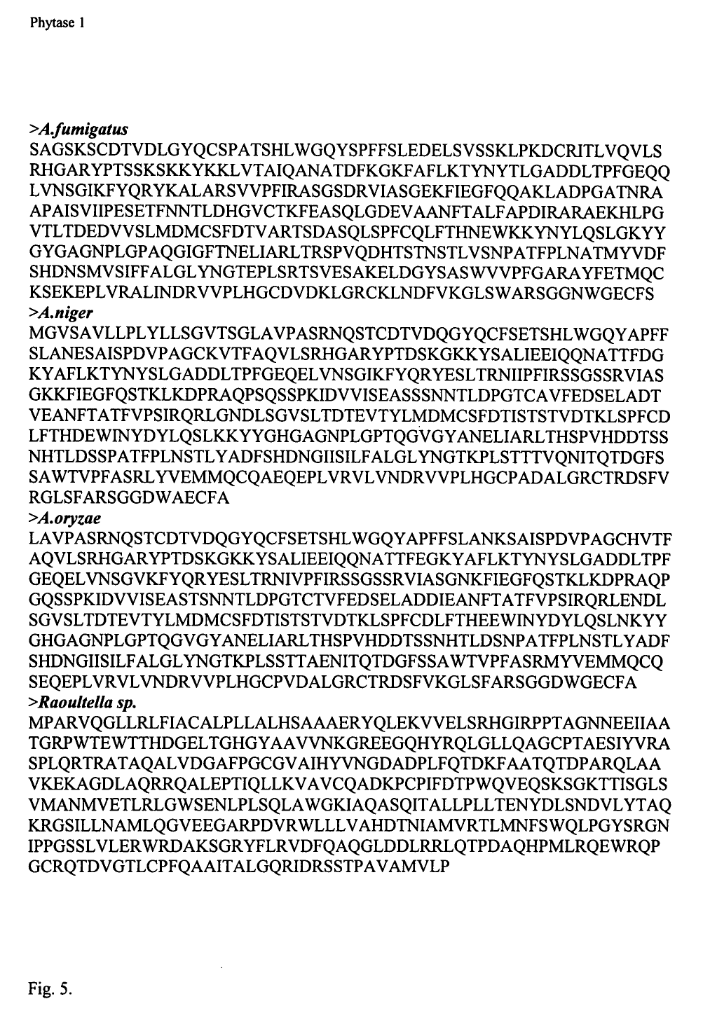

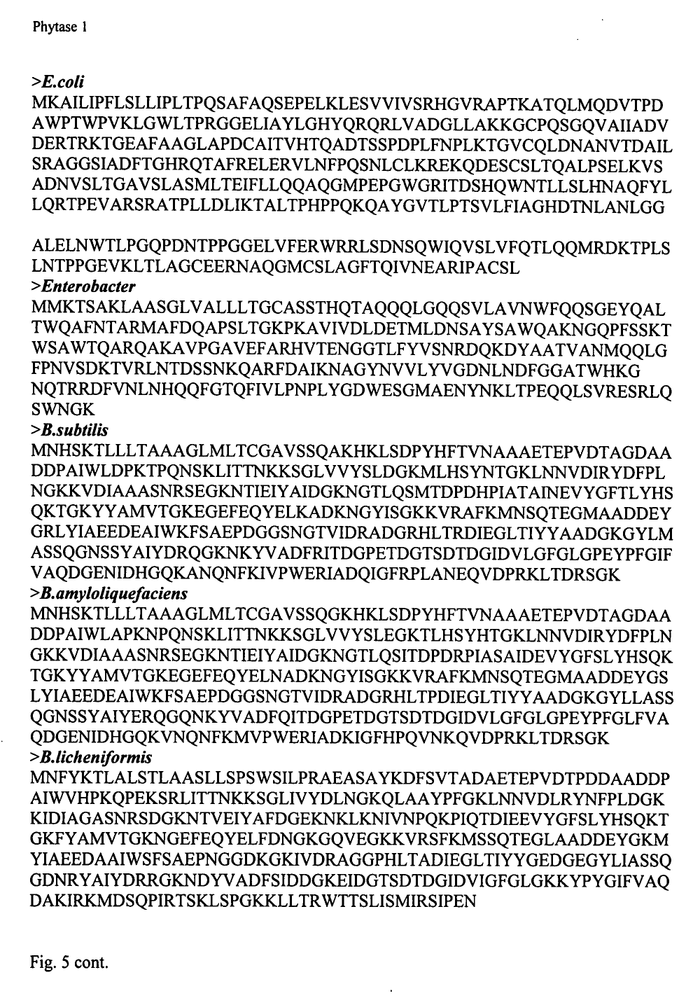

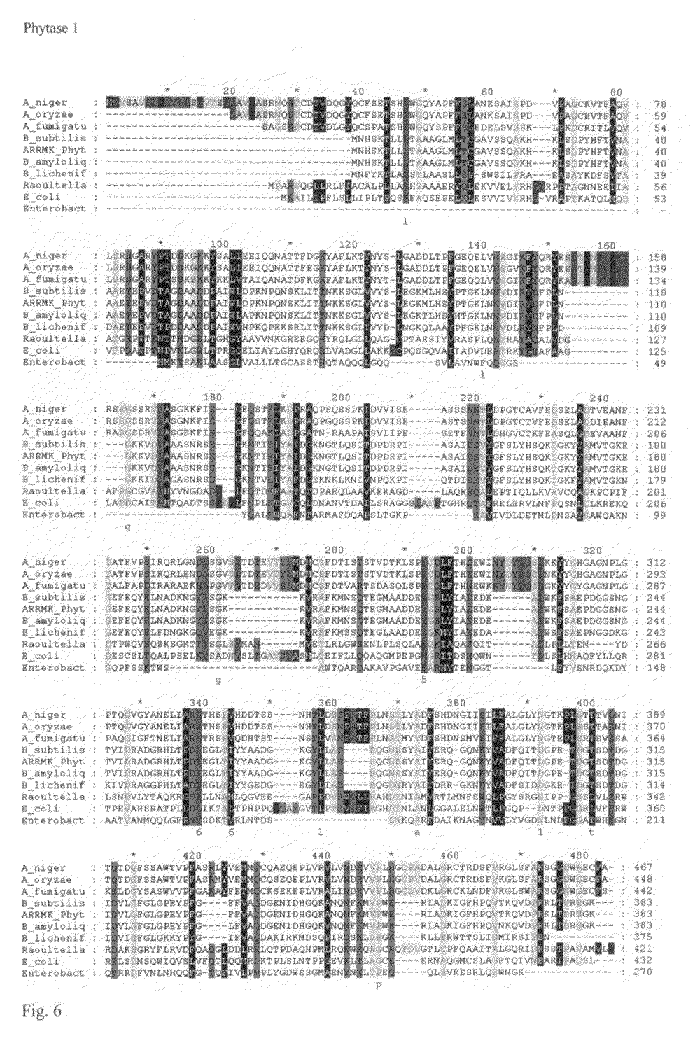

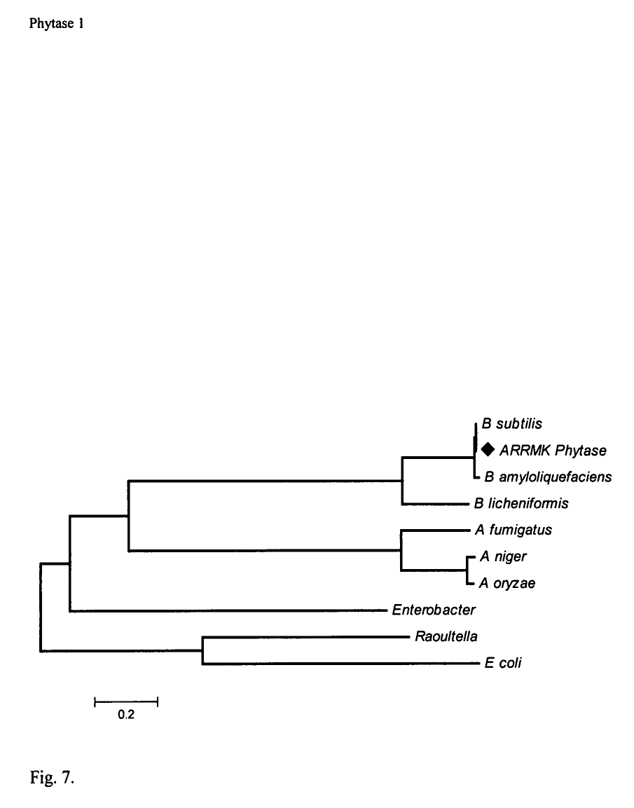

A standard protocol for phytase assay was followed as shown in FIG. 2 by taking 200 μl of crude sample of phytase into 10 mL test tubes and incubated at 37° C. water bath for 5 min. 200 μl of 1.25% (wt/vol) sodium phytate in selected buffer and pH was used for enzymatic hydrolysis of phytate, and incubated for 15 min at 37° C. Reaction was terminated by adding 400 μl of 15% trichloroacetic acid. The mixture was centrifuged at 2,000 g for 10 min and 200 μl of supernatant was added to 1.8 mL of double distilled water. 2 mL of fresh color reagent (3 vol. of 1M H₂SO₄+1 vol. of 2.5% ammonium molybdate+1 vol. of 10% ascorbic acid) was added and mixed well. The mixture was incubated 50° C. for 15 min and left at room temperature for 2-3 min. The absorbance was read at 820 nm, using water as the blank and the series diluted potassium phosphate solutions as standards. Phytase activity was calculated per ml of culture and expressed as Units/Litre. One unit of phytase is defined as the amount of enzyme required to release 1 u mol of inorganic phosphate/min from sodium phytate at 37° C.

### Biochemical Properties of the Recombinant Phytase:

The molecular weight of the novel recombinant phytase was determined to be 41790.95.

The isolectric point of the enzyme was defined to be 4.79

Aminoacid composition of the enzyme is analyzed in Table 1. below.

TABLE 1
Amino acid composition of the recombinant phytase

| | No. | Percent |
|---|---|---|
| Non-polar: | | |
| A | 35 | 9.11 |
| V | 17 | 4.43 |
| L | 24 | 6.25 |
| I | 22 | 5.73 |
| P | 17 | 4.43 |
| M | 7 | 1.82 |
| F | 13 | 3.39 |
| W | 3 | 0.78 |
| Polar: | | |
| G | 38 | 9.90 |
| S | 25 | 6.51 |
| T | 24 | 6.25 |
| C | 1 | 0.26 |
| Y | 20 | 5.21 |
| N | 20 | 5.21 |
| Q | 15 | 3.91 |
| Acidic: | | |
| D | 33 | 8.59 |
| E | 22 | 5.73 |
| Basic: | | |
| K | 29 | 7.55 |
| R | 10 | 2.60 |
| H | 8 | 2.08 |

The protein has no rigid secondary structure due to lack of cysteine—cysteine covalent bonds. The enzyme is a heat stable protein

Phytase Enzyme Assay:

Enzyme kinetics studies performed on purified enzyme samples were accomplished by the assay of inorganic phosphate liberated from corn phytic acid. Exhaustive phytate hydrolysis was accomplished by incubating 1.25% phytic acid with enzyme (U/ml) in 0.2 M sodium citrate, pH 5.5, at 37° C. Standard enzyme kinetics reactions were carried out for 15 min at 37° C. in 1.25% (wt/wt) phytic acid. The reaction was quenched by the addition of an equal volume of 15% (wt/wt) trichloroacetic acid. Centrifugation is done for 10 min at 2000×g. Color reagent (1 ml) was added to 0.2 ml of supernatant, incubated at 50° C. for 15 min. The absorbance was measured at 820 nm. The color reagent was composed of 1 M sulfuric acid, 2.5% (wt/vol) hepta-ammonium molybdate, 10% ascorbic acid in a ratio of 3:1:1 and was prepared fresh daily. Quantitation was based on a standard curve generated with a 9 mM sodium monobasic phosphate standard. One unit is defined as 1 umol of inorganic phosphate released per min with 1.25% phytic acid in 0.2 M sodium citrate, pH 5.5, at 37° C. The phytase activity is calculated to be ˜4,11,371 U/L broth.

TABLE 2
Concentration of Potassium phosphate used for Phosphate
standard graph

| Dilution of 9 mM | Phosphate |
|---|---|
| Phosphate stock | Concentration (μM) |
| 5:195 | 225 |
| 10:190 | 450 |
| 15:185 | 675 |
| 20:180 | 900 |
| 25:175 | 1125 |

Thermo Stability Measurement:

Phytase samples were dissolved at 100U per ml in 0.2 M sodium citrate, pH 5.5. (Randy and Berka et al., 1998) Twenty-microliter aliquots of each enzyme solution were incubated for 20 min in a water bath at 37, 45, 50, 55, 60, 65, 70, and 75° C. After the heat treatment, the samples were stored at 0° C. until activity assays were performed. The data represented here is derived from experiments conducted in triplicates (Table 3 and FIG. 14).

TABLE 3
Determination of optimal temperature for phytase activity at pH5.5

| Temperature (° C.) | Phytase (U/ml) | Phosphate released (μM) |
|---|---|---|
| 37 | 8,079 | 3030 |
| 45 | 14,399 | 5400 |
| 55 | 8,986 | 3370 |
| 65 | 9,279 | 3480 |
| 75 | 10,479 | 3930 |

pH Activity Measurement:

To attain a buffering range between pH 2 and 8, different buffers were made. This process was repeated for every pH units through pH 2. The activity of phytase is highest at pH 7 (Table 4 and FIG. 15).

TABLE 4
Determination of optimal pH for phytase activity

| pH | Phytase (U/ml) | Phosphate released (μM) |
|---|---|---|
| 2 | 5999 | 2250 |
| 2.5 | 5999 | 2250 |
| 4.5 | 5999 | 2250 |
| 5.5 | 7199 | 2700 |
| 7.0 | 8399 | 3150 |
| 8.0 | 6599 | 2745 |

### EXAMPLE 10

### Use of the novel recombinant phytase in industrial applications

Food Industry:

A diet, rich in cereal fibers, legumes and soy protein results in an increased intake of phytate. Vegetarians, eldery people consuming unbalanced food with high amounts of cereals, people in undeveloped countries who eat unleavened bread and babies eating soy-based infant formulas take in large amounts of phytate (Simell et al., 1989). Undigested phytate in the small intestine negatively affects the absorption of zinc, calcium and magnesium.

The novel recombinant phytase according to the current invention is suitable to be used as food additive to digest phytate in the digestive track of human beings or animals. Characteristics that make the enzyme according to this invention suitable for such use, is the rather high activity at pH range of 5.5 to 7.5 which is the pH of small intestine and the high activity at temperature range between 37° C. and 41° C. which is the temperature in the digestive tract.

Pulp and Paper Industry:

It has been speculated that the removal of plant phytic acid might be important in the pulp and paper industry. A thermostable phytase could have potential as a novel biological agent to degrade phytic acid during pulp and paper processing. The enzymatic degradation of phytic acid would not produce carcinogenic and highly toxic by-products. Therefore, the exploitation of phytases in the pulp and paper process could be environmentally friendly and would assist in the development of cleaner technologies (Liu et al., 1998).

The novel recombinant phytase according to the current invention is suitable to be used in paper and pulp industry. Especially the thermostability of the novel phytase is a key in this application as the temperatures used in paper and pulp production steps are usually high.

Although the invention has been described in some detail by way of illustration and example for purposes of clarity of understanding, it will be clear to one skilled in the art that certain changes and modifications may be practiced within the scope of the appended claims.

What is claimed is:

1. An isolated nucleic acid, which encodes a phytase enzyme having an aminoacid sequence essentially according to SEQ ID NO:2, and said nucleic acid being essentially according to SEQ ID NO:1.

2. The nucleic acid according to claim 1, wherein the nucleic acid is the coding sequence of a phytase coding gene obtainable from Bacillus subtilis strain ARRMK33.

3. An expression vector comprising:
a leftward promoter pL of coliphage A;
a c1857 allele coding for a thermolabile repressor protein; and
coding sequence of phytase enzyme downstream from the pL promoter, said sequence being obtainable from B. subtilis AMRKK33, and further being essentially according to SEQ ID NO:1.

4. A prokaryotic host cell transformed with the expression vector of claim 3.

5. The prokaryotic host cell according to claim 4, wherein the host cell is co transformed with chaperone vector pTf16.

6. A method to produce recombinant phytase enzyme in microbial cells, said method comprising the steps of:
a. Providing an expression vector according to claim 3;
b. Transforming E. coli host cells with the vector
c. Co transforming E. coli cells containing the expression vector with chaperone vector pTF16;
d. Culturing the transformed cells in culture media;
e. Increasing temperature of the culture media to about 42 C thereby denaturalizing repressor protein expressed by c1857 allele;
f. Further culturing the cells; and
g. Purifying recombinant phytase from soluble fraction of the culture.

7. A recombinant phytase enzyme produced and purified according to the method of claim 6, wherein the enzyme has an amino acid sequence essentially according to SEQ ID NO:2.

8. The recombinant phytase enzyme of claim 7, wherein the enzyme is further characterized by that it is thermostable up to about 70° C., its pH optimum is between 5,5 and 7,5.
